(19) United States
(12) Patent Application Publication
(10) Pub. No.: US 2024/0199863 A1
(43) Pub. Date: Jun. 20, 2024

(54) Rubber Component With Reduced Drug Potency Loss for Syringe Stopper and Container Closure System Applications

(71) Applicant: Becton, Dickinson and Company, Franklin Lakes, NJ (US)

(72) Inventors: James Joseph Semler, Randolph, NJ (US); Chad H. Smith, Phillipsburg, NJ (US); Douglas P. Cusato, Washington Township, NJ (US)

(21) Appl. No.: 18/557,204

(22) PCT Filed: Apr. 28, 2022
(86) PCT No.: PCT/US2022/026668

§ 371 Date: Oct. 25, 2023

Related U.S. Application Data
(60) Provisional application No. 63/181,283, filed on Apr. 29, 2021.

(51) Int. Cl. C08L 9/06; A61M 5/315

(52) U.S. Cl. CPC C08L 9/06; A61M 5/31511; C08L 2203/02

(57) ABSTRACT

Provided herein are elastomeric materials for use in medical devices and methods of making elastomeric materials, the elastomeric materials including a rubber composition and a phase-separating composition. Also provided herein are medical devices including elastomeric materials, the elastomeric materials including a rubber composition and a phase-separating composition.

### CROSS-REFERENCE TO RELATED APPLICATION

The present application claims priority to U.S. Provisional Application Ser. No. 63/181,283, entitled “Rubber Component with Reduced Drug Potency Loss for Syringe Stopper and Container Closure System Applications”, filed Apr. 29, 2021, the entire disclosure of which is herein incorporated by reference.

### BACKGROUND OF THE INVENTION

### Field of the Invention

The present invention relates to improved rubber materials for medical applications. More particularly, the invention relates to the use of a viscous, low surface energy minor phase-separating composition in a major-phase rubber material for stoppers, septums, and other elastomeric, rubber-based medical device components.

### Description of Related Art

Medical devices that are used to hold or deliver therapeutic compositions are typically produced from or otherwise include materials that can undesirably interact with the therapeutic composition. Similarly, medical devices that are used to collect or hold samples, for example blood samples, are typically produced from or otherwise include materials that can undesirably interact with blood components or analytes of interest in the sample. Undesired interactions can include, for example, adsorption or absorption of a compound or analyte of interest to a component of the medical device, thus affecting potency of a therapeutic substance or interfering with analysis of a sample.

Typical approaches for dealing with such materials include manufacturing the medical devices, or components thereof, from materials that are more inert. However, such manufacturing methods can greatly increase the complexity and cost of manufacturing, particularly for mass-produced devices such as blood collection containers and pre-filled syringes.

Another approach to address undesirable interactions is to coat components of medical devices with an inert coating. However, as described above, adding a coating to various components of medical devices, particularly mass-produced devices, greatly increases complexity and cost. Further, not all coatings will be suitably inert for all possible uses.

Accordingly, a need exists in the art for a material for use in medical devices that can provide a desired lack of reactivity or interaction with a component of a substance held in or otherwise contacted by the medical device.

### SUMMARY OF THE INVENTION

Provided herein is an elastomeric material for forming a stopper for a syringe. The elastomeric material includes a rubber composition and a phase-separating composition, and the phase-separating composition migrates within the rubber composition to form a drug-absorption resistant barrier layer.

Also provided herein is a method of preparing an elastomeric material including the steps of preparing a mixture of a major phase comprising a rubber composition and a minor phase comprising a phase-separating composition and allowing the minor phase of the mixture to accumulate at a surface of the elastomeric material.

Also provided herein is a medical device including a barrel defining a chamber for receiving a solution therein, and an elastomeric stopper. The elastomeric stopper includes a rubber composition and a phase-separating composition, and the phase-separating composition is arranged at a surface of the stopper arranged to contact the solution held within the chamber.

Also provided herein is an elastomeric material including a rubber composition and a phase-separating composition.

In accordance with an embodiment of the present invention, an elastomeric material for forming a stopper for a syringe includes a rubber composition and a phase-separating composition, wherein the phase-separating composition migrates within the rubber composition to form a drug-absorption resistant barrier layer. Optionally, the rubber composition may be a natural rubber. In other configurations, the rubber composition is a synthetic rubber. The rubber composition may be one or more of polyisoprene, polybutadiene, styrene ethylene butylene styrene, epichlorohydrin, olefin block copolymer, bromobutyl rubber, silicone, and/or styrene-butadiene.

In certain configurations, the phase-separating composition has a lower surface energy than the rubber composition. In other configurations, the phase-separating composition is one or more of paraffin wax, polyethylene wax, and silicone. Optionally, the phase-separating composition is an olefin. In certain configurations, the phase-separating composition has a melting point of from about −80° C. to about 180° C., which may optionally be from about 37° ° C. to about 180° C. The phase-separating composition may be included in the elastomeric material at from above 0 to about 20 parts per hundred rubber (phr), and may optionally between above 0 phr and to about 10 phr, such as above 0 and to about 1 phr, or between about 1 phr to about 6 phr.

In accordance with another embodiment of the present invention, a method of preparing an elastomeric material includes preparing a mixture of a major phase including a rubber composition and a minor phase including a phase-separating composition. The method further includes the step of allowing the minor phase of the mixture to accumulate at a surface of the elastomeric material. The method further includes the step of preparing the mixture including mixing the major phase and the minor phase until the minor phase is uniformly distributed within the major phase.

In certain configurations, the method further includes the step of heating the mixture. Optionally, the rubber composition may be a natural rubber. Alternatively, the rubber composition may be a synthetic rubber. The rubber composition may be one or more of polyisoprene, polybutadiene, styrene ethylene butylene styrene, epichlorohydrin, olefin block copolymer, bromobutyl rubber, silicone, and/or styrene-butadiene. The phase-separating composition may have a lower surface energy than the major phase rubber composition. Optionally, the phase-separating composition may include one or more of paraffin wax, polyethylene wax, and silicone. In certain situations, the phase-separating composition is an olefin.

In certain configurations, the phase-separating composition has a melting point of from about −80° C. to about 180° C., which may optionally be from about 37° C. to about 180° C. The method may also include molding the mixture to produce an elastomeric material for a medical device. The molding may be selected from the group consisting of compression molding, injection molding, reactive injection molding, and/or liquid injection molding. In certain configurations, the minor phase may be included in from above 0 to about 20 phr, and optionally between above 0 phr and to about 10 phr, such as above 0 and to about 1 phr, and optionally between about 1 phr to about 6 phr.

In accordance with another embodiment of the present invention, a medical device includes a barrel defining a chamber for receiving a solution therein, and an elastomeric stopper including a rubber composition and a phase-separating composition, wherein the phase-separating composition is arranged at a surface of the stopper arranged to contact the solution held within the chamber.

In certain configurations, the medical device is a syringe. In other configurations, the medical device is a blood collection tube.

In accordance with another embodiment of the present invention, an elastomeric material includes a rubber composition and a phase-separating composition.

Optionally, the phase-separating composition includes one or more of paraffin wax, polyethylene wax, and silicone. The phase-separating composition may be an olefin.

In accordance with yet another embodiment of the present invention, a stopper made according to the above-described methods has a drug-absorption resistant barrier later at a liquid-contacting surface of the stopper.

### BRIEF DESCRIPTION OF THE DRAWINGS

FIG. 1 is a schematic representation of a process for making an elastomeric material according to one non-limiting embodiment or aspect of the present invention.

FIG. 2 is an exploded perspective view of a medical device including an elastomeric material according to one non-limiting embodiment or aspect of the present invention.

FIG. 3 is a graphical representation of the recovery of fentanyl from solution vs. additive content in rubber measured over days of storage in medical devices according to non-limiting embodiments or aspects of the present invention.

FIG. 4 is a graphical representation of fentanyl potency over time in standard medical devices and those according to non-limiting embodiments or aspects of the present invention.

FIG. 5 is a graphical representation of the recovery of fentanyl from solution vs. time in a device utilizing a parylene coating.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention can assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The figures accompanying this application are representative in nature, and should not be construed as implying any particular scale or directionality, unless otherwise indicated.

The use of numerical values in the various ranges specified in this application, unless expressly indicated otherwise, are stated as approximations as though the minimum and maximum values within the stated ranges are both preceded by the word “about”. In this manner, slight variations (e.g., ±10%) above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

Provided herein are elastomeric materials for use in medical devices. These elastomeric materials exhibit lower interaction and/or reactivity with various components in compositions received or stored in the medical devices. As used herein, “interaction” or “reactivity” mean any interaction between a composition received or stored within a medical device and the elastomeric material. Non-limiting examples of interactions or reactions include adsorption or absorption of the composition to the elastomeric material, precipitation of the composition out of solution, degradation of the composition, and contamination of the composition, for example by leeching of components of the elastomeric material into the composition, and the like.

Elastomeric materials according to the present invention may include a non-rubber elastomer composition and a phase-separating composition, and/or a rubber composition and a phase-separating composition. The non-rubber elastomer composition may include, without limitation, polypropylenes, polyethylenes, polycarbonates, and the like. The rubber composition can be a natural rubber, a synthetic rubber, or combinations thereof. In non-limiting embodiments or aspects the rubber composition selected from one or more of butyl-based rubbers, for example those based on a polyisobutylene skeleton such as butyl rubber (isobutylene-isoprene copolymer), chlorinated butyl rubbers, brominated butyl rubbers, bromides of an isobutylene-paramethylstyrene copolymer, polyisoprene rubbers, polybutadiene rubbers, styrene ethylene butylene styrene (SBS) rubbers, epichlorohydrin rubbers, styrene-butadiene (SBR) rubbers, and combinations thereof. Those of skill in the art will appreciate that various types of materials commonly used in manufacturing stoppers, septums, and other elastomeric components of medical devices can be utilized in the present invention.

The elastomeric material provided herein also includes a phase-separating composition. As used herein, the term “phase-separating composition” means a composition in a mixture that will preferentially diffuse to the external interface over time. Those of skill in the art will appreciate that suitable phase-separating compositions will vary based on the elastomer (rubber or non-rubber) that is utilized in the elastomeric material, and that a consideration for selecting the phase-separating composition is that the phase-separating composition, relative to the rubber/non-rubber elastomer, will not result in a negative free energy of mixing and will maintain a positive curvature over the entire concentration range. Such separation can be based on differences in chemical properties between the phase-separating composition and other compositions in the mixture, for example, hydrophobicity/hydrophilicity. Suitable phase-separating compositions are those compositions that are lower surface energy than the matrix, non-reactive, and differ in at least one chemical characteristics from the rubber composition utilized in the elastomeric material. The phase-separating composition utilized in the elastomeric material can be selected based on the rubber composition that is utilized, but can include, for example and without limitation, waxes, such as paraffin waxes and polyethylene oxide waxes, silicone-based compositions such as silicone oils, mineral oils, and the like. In non-limiting embodiments or aspects, the phase-separating composition is an olefin, an olefin-based composition, or a composition that exhibits olefin-like chemistry.

In non-limiting embodiments or aspects the phase-separating composition has a lower surface energy than the rubber composition utilized in the elastomeric material, measured using water contact angle. In some non-limiting embodiments or aspects the phase-separating composition has a surface energy of between about 15 mJ/m² and about 30 mJ/m². In non-limiting embodiments or aspects, the phase-separating composition has a melting point of between about −80° C. and about 180° C., optionally between about 37° C. and about 180° C., all subranges therebetween inclusive. Those of skill in the art will appreciate that suitable phase-separating compositions can be selected based on their respective physical and/or chemical properties.

As described below, elastomeric materials prepared according to the invention described herein include a major phase of a rubber composition and a minor phase including a phase-separating composition. Thus, the phase-separating composition is included in the elastomeric material in a smaller amount than the rubber composition. In non-limiting embodiments or aspects, the phase-separating composition is included in an amount of between an amount above 0 and about 20 parts per hundred of the rubber composition (phr), optionally between an amount above 0 and to about 10 phr, all subranges therebetween inclusive. In some non-limiting embodiments or aspects the phase-separating composition is included in the elastomeric material in an amount between above 0 and about 1 phr. In other non-limiting embodiments or aspects, the phase-separating composition is included in the elastomeric material in an amount between about 1 and about 6 phr. Those of skill in the art will appreciate that the selection of phase-separating composition can inform the amount to be included in the elastomeric material.

In addition to the choice of rubber composition, in non-limiting embodiments or aspects the identity of the composition that is or is likely to be received or stored within the medical device can be used as a basis for selecting the phase-separating composition. In non-limiting embodiments or aspects of the present invention the phase-separating composition is selected to differ in at least one chemical property from the composition that is or is likely to be received or stored within the medical device, thereby reducing or preventing interactions between the composition received or stored within the medical device and the elastomeric material.

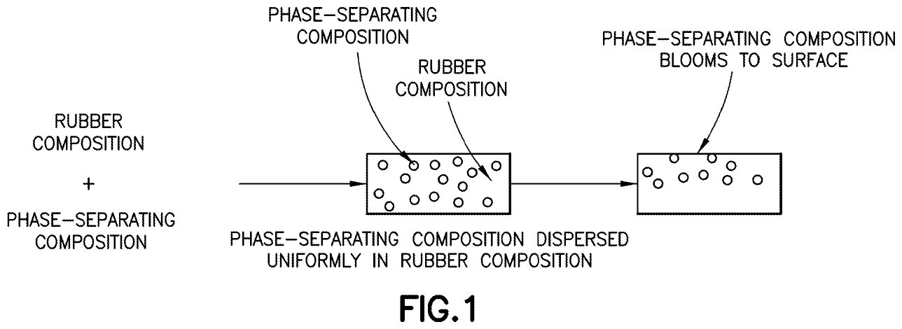

Also provided herein are methods of preparing elastomeric materials for use in medical devices. The method includes the steps of preparing a mixture of a major phase and a minor phase. The major phase includes a rubber composition, and the minor phase includes a phase-separating composition. The rubber composition and the phase-separating composition can include those identified herein above, in amounts likewise identified herein above. With reference to FIG. 1, the method further includes the step of allowing the phase-separating composition to accumulate at a surface of the elastomeric material. The time required for accumulation at the surface, or blooming, of the phase-separating composition can be adjusted, but advantageously occurs during the manufacturing cycle. FIG. 1 illustrates a non-limiting embodiment or aspect of the present method, in which the major phase (rubber composition) and minor phase (phase-separating composition) are mixed, such that the phase-separating composition is dispersed uniformly within the major phase rubber composition matrix. While not wishing to be bound by the theory, it is believed that, over time because of the difference in chemistries between the rubber composition and the phase-separating composition, the phase-separating composition will “bloom”, or accumulate, at a surface of the elastomeric material. This blooming allows for the elastomeric material to be oriented or arranged such that the phase-separating composition, which as described above is less reactive and/or exhibits less interaction with components within a composition received or stored within a medical device, contacts that composition rather than the major phase rubber composition. The present invention is advantageous at least because it allows for a single step of preparing an elastomeric material for use in a medical device. Whereas, for example, stoppers with coatings or films, or two-piece stoppers, exist, and traditional stoppers (without additives/coatings) can be treated by washing, vacuum baking, autoclaving, and the like, those products and treatments require multiple manufacturing steps, increasing complexity, time, and cost. In contrast, the present elastomeric materials can be prepared in a single step, mixing together a major phase of a rubber composition and a phase-separating composition, then allowing the minor phase (phase-separating composition) to migrate, or bloom, and form a barrier. In non-limiting embodiments or aspects, the mixture is heated, to accelerate migration/blooming of the minor phase phase-separating composition within the major phase rubber composition. Those of skill in the art will appreciate that the temperature used will depend on the phase-separating composition that is used in the elastomeric material disclosed herein.

In non-limiting embodiments or aspects, the method described herein further includes the step of molding the mixture to produce an elastomeric material for a medical device. As described above, the blooming, or accumulation, of the phase-separating composition allows for elastomeric components having a directionality to be prepared. These components can be molded in a manner such that the phase-separating composition, which as shown in FIG. 1 forms a layer at a surface of the elastomeric material, can be arranged to be the portion of the elastomeric material that comes into contact with a composition received or stored within the medical device in which the elastomeric material is incorporated or included. Those of skill in the art will appreciate that any suitable molding process can be utilized. In non-limiting embodiments or aspects the elastomeric material is molded using compression molding or injection molding, for example, and without limitation, reactive injection molding or liquid injection molding. In further non-limiting embodiments or aspects, the molded elastomeric material is cross-linked or otherwise polymerized such that an elastomeric component is formed.

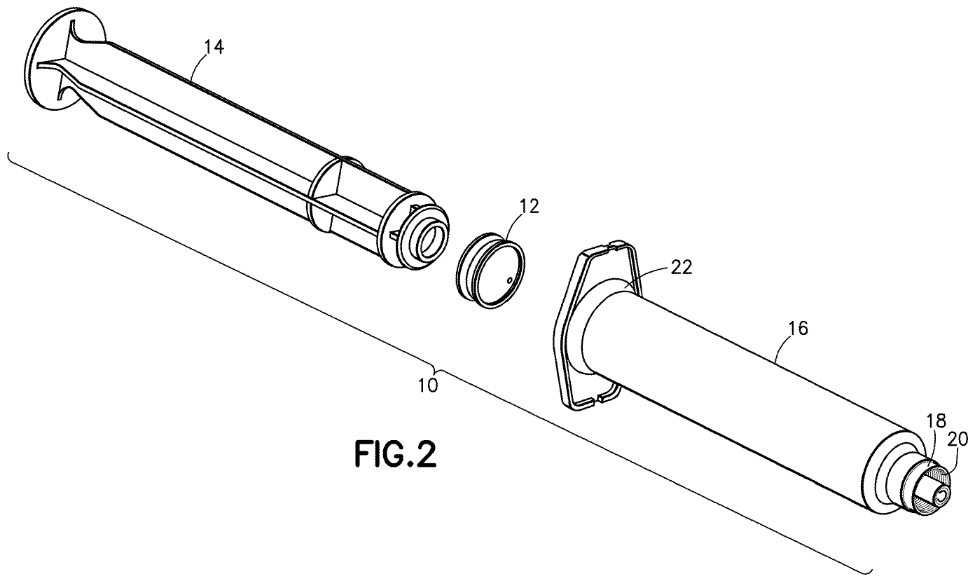

Also provided herein are medical devices including an elastomeric component including the described elastomeric materials. Any medical device that utilizes an elastomeric component is within the scope of this disclosure, and such devices can include, for example and without limitation, syringes, specimen collection containers, vials for storage of pharmaceutical compositions, IV bags, medical pump pistons, IV tubing, and the like. Those of skill in the art will appreciate that any medical device that could benefit from a lower interaction between compositions received or stored therein and an elastomeric component falls within the scope of the present disclosure. In some non-limiting embodiments or aspects the medical device includes a barrel defining a chamber for receiving or storing a composition therein and one or more elastomeric stoppers or septums, one or more of the elastomeric stopper(s) or septum(s) being formed of an elastomeric material including a rubber composition and a phase-separating composition as described herein. In non-limiting embodiments or aspects, such as illustrated in FIG. 2, the medical device is a syringe (10) including a barrel (16), a proximal end (18), a distal end (22), a plunger (14), and an elastomeric stopper (12), the elastomeric stopper being formed of an elastomeric material as described herein. Syringe barrel (16) and plunger (14) can be formed out of any suitable materials, such as glass and/or plastic, as is known in the art. The syringe (10) can include a needle (not shown) fixedly or removably attached, or a luer connection (20) to allow for removable connection to needles and other fluid transfer devices as is known in the art. In other non-limiting embodiments or aspects the medical device is a specimen collection container, for example a blood collection container, including a stopper formed of an elastomeric material as described herein.

### EXAMPLES

### Example 1

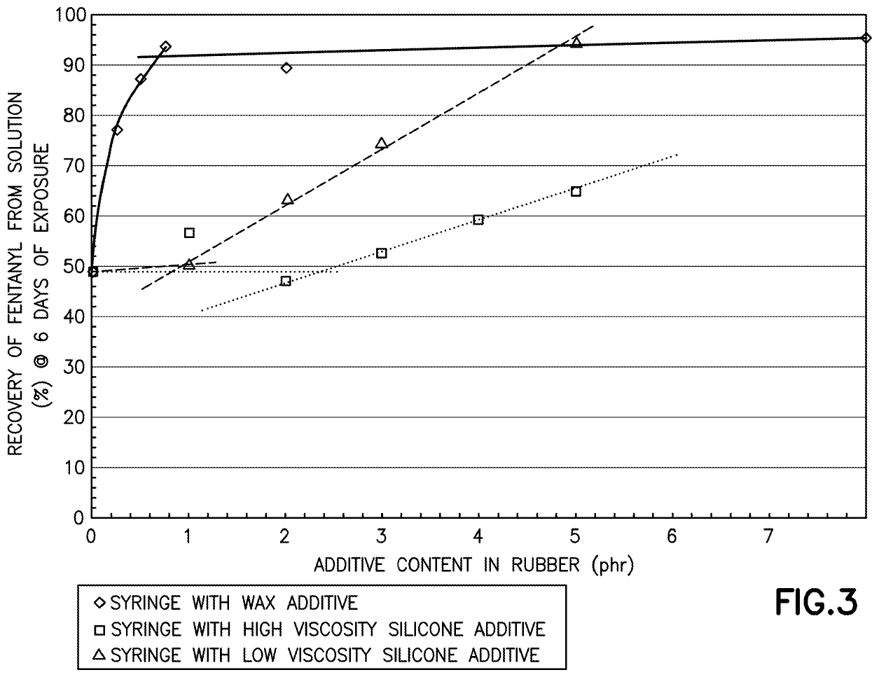

Elastomeric compositions including rubber and a phase separating material at from 0-8 parts per hundred of rubber (phr) were generated, and were placed into contact with a fentanyl solution for six days. Syringes comprised of polypropylene barrels, polypropylene plunger rods, and SBR rubber stoppers with the different additives were utilized. Stopper samples were prepared under normal compression molding process conditions. Recovery of fentanyl from solution was measured after the six-day incubation/storage. FIG. 3 shows results of the experiment for gamma-sterilized materials (gamma irradiation in the range of 20-40 kGy) including paraffin wax (♦), a low viscosity silicone (▴, Dow-Corning DC360, 100 cSt), and a high viscosity silicone (▪, Dow-Corning DC360, 12,500 cSt). As shown in FIG. 3, fentanyl recovery was high (>90% recovery) following incubation/storage with an elastomeric material including a paraffin wax phase-separating composition with at least 0.75 phr. As also shown in FIG. 3, recovery of fentanyl increased as concentration of silicone oil in the elastomeric composition increased, with low-viscosity silicone exhibiting fewer interactions/greater recovery.

### Example 2

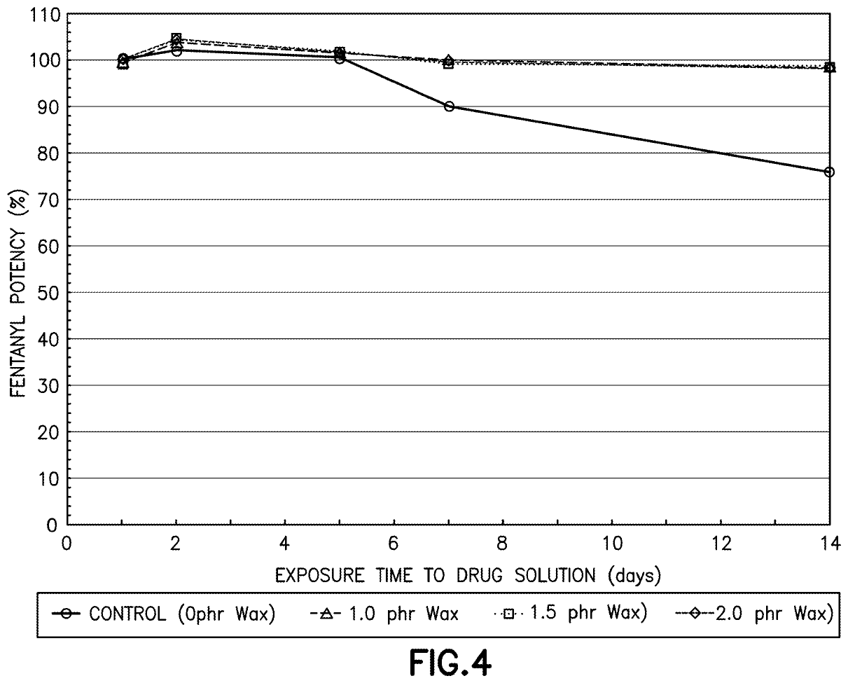

Elastomeric compositions including SBR rubber alone or with wax at concentrations of 1, 1.5, and 2 phr were generated. The elastomeric materials were formed into stoppers under standard compression molding conditions (sterilization not performed) and included in syringes having polypropylene barrels. 0.3 mL of a fentanyl solution (5 μg/mL, pH 4.09) was introduced to the syringe barrels, and fentanyl potency (FIG. 4) was measured at 1, 2, 5, 7, and 14 days. In FIG. 4, at all concentrations of wax used, potency of fentanyl relative to the control (no wax included in the elastomeric composition from which the stopper was formed) was increased. Specifically, in all conditions including wax (1, 1.5, and 2 phr), >90% potency of fentanyl was seen through 14 days of exposure, while the control (0 phr of wax) showed less than 90% potency of fentanyl after 7 days of exposure.

### Comparative Example

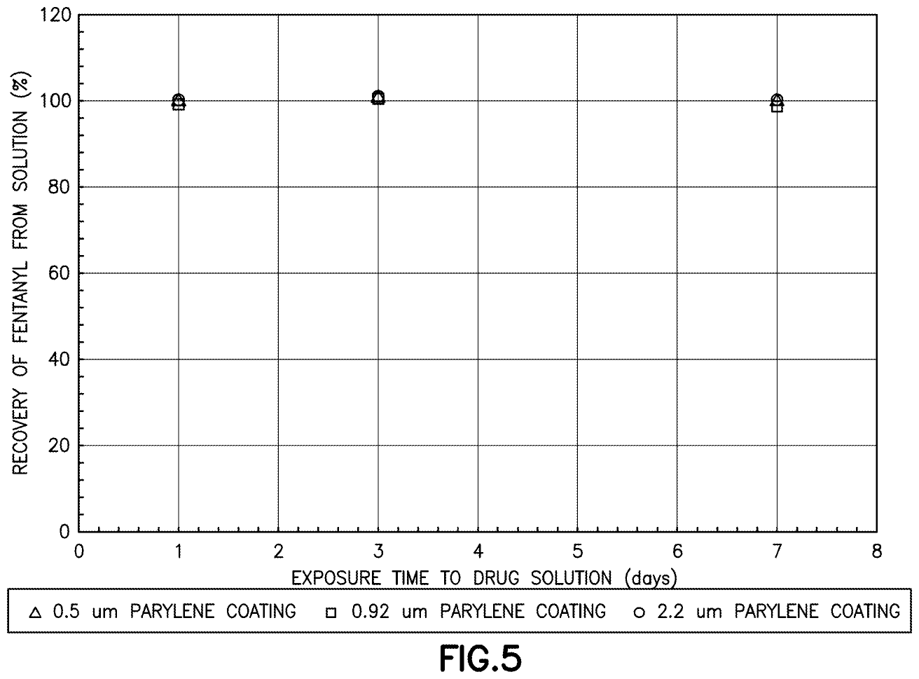

Elastomeric compositions including a parylene coating were prepared for comparison to materials as disclosed herein. As seen in FIG. 5, at all time points over the course of eight days, fentanyl recovery from solution was 100% for the parylene-coated stoppers. Thus, as can be ascertained from Example 1 and the present example, compositions as disclosed herein provide equivalent performance in terms of recovery of a therapeutic composition from solution, with savings in terms of cost and manufacturing time.

While the present invention has been described in terms of the above detailed description, those of ordinary skill will understand that alterations may be made within the spirit of the invention. Accordingly, the above should not be considered limiting, and the scope of the invention is defined by the appended claims.

1. An elastomeric material for forming a stopper for a syringe comprising a rubber composition and a phase-separating composition, wherein the phase-separating composition migrates within the rubber composition to form a drug-absorption resistant barrier layer.

2. The elastomeric material according to claim 1, wherein the rubber composition is a natural rubber.

3. The elastomeric material according to claim 1, wherein the rubber composition is a synthetic rubber.

4. The elastomeric material according to claim 1, wherein the rubber composition is one or more of polyisoprene, polybutadiene, styrene ethylene butylene styrene, epichlorohydrin, olefin block copolymer, bromobutyl rubber, silicone, and styrene-butadiene.

5. The elastomeric material according to claim 1, wherein the phase-separating composition has a lower surface energy than the rubber composition.

6. The elastomeric material according to claim 1, wherein the phase-separating composition is one or more of paraffin wax, polyethylene wax, and silicone.

7. The elastomeric material according to claim 1, wherein the phase-separating composition is an olefin.

8. The elastomeric material according to claim 1, wherein the phase-separating composition has a melting point of from about −80° C. to about 180° C., optionally from about 37° C. to about 180° C.

9. The elastomeric material according to claim 1, wherein the phase-separating composition is included in the elastomeric material at from above 0 to about 20 parts per hundred rubber (phr), optionally between above 0 phr and to about 10 phr, optionally above 0 and to about 1 phr, optionally between about 1 phr to about 6 phr.

10.-22. (canceled)

23. A medical device comprising:
a barrel defining a chamber for receiving a solution therein; and
an elastomeric stopper comprising a rubber composition and a phase-separating composition, wherein the phase-separating composition is arranged at a surface of the stopper arranged to contact the solution held within the chamber.

24. The medical device according to claim 23, wherein the medical device is a syringe.

25. The medical device according to claim 23, wherein the medical device is a blood collection tube.

26. An elastomeric material comprising:
a rubber composition; and
a phase-separating composition.

27. The elastomeric material according to claim 26, wherein the phase-separating composition is one or more of paraffin wax, polyethylene wax, and silicone.

28. The elastomeric material according to claim 26, wherein the phase-separating composition is an olefin.

29. A stopper made of the elastomeric material according to claim 26, wherein the drug-absorption resistant barrier layer is at a liquid-contacting surface of the stopper.
